(19) United States
(12) Patent Application Publication
(10) Pub. No.: US 2010/0215625 A1
(43) Pub. Date: Aug. 26, 2010

(54) EFFICIENT PREPARATION OF NAPHTHOQUINONE ANTICANCER ACTIVE INGREDIENTS

(75) Inventors: Akira Iida, Kyoto-shi (JP); Ryuji Takeda, Kizugawa-shi (JP); Masafumi Kaneko, Takasaki-shi (JP); Harukuni Tokuda, Kyoto-shi (JP)

Correspondence Address: HAMRE, SCHUMANN, MUELLER & LARSON, P.C., P.O. BOX 2902, MINNEAPOLIS, MN, 55402-0902, US

(73) Assignee: TAHEEBO JAPAN CO., LTD., Osaka-shi (JP)

(21) Appl. No.: 12/605,684

(22) Filed: Oct. 26, 2009

(30) Foreign Application Priority Data
Feb. 25, 2009 (JP) 2009-42624
Sep. 1, 2009 (JP) 2009-201563

(51) Int. Cl. A61K 36/185; C12N 5/04; C07D 307/92

(52) U.S. Cl. 424/93.7; 435/420; 549/458; 435/410

(57) ABSTRACT

One of the problems to be resolved by the present invention is to stably and sustainably produce active ingredients contained in Bignoniaceous plants of which a mass production is difficult in a conventional manner. The present invention relates to a method for efficiently preparing an anticancer active ingredient NQ801 by a cell cultivation of Bignoniaceous plants under specific culture conditions. An ingredient-production system in the present invention has an anticancer activity.

### TECHNICAL FIELD

The present invention relates to a method for efficiently preparing anticancer active ingredients by a cell cultivation of Bignoniaceous plants.

The present invention also relates to a food product containing clumps of cells or callus of said plants comprising anticancer active ingredients produced by the cell cultivation.

### BACKGROUND ART

Bignoniaceous Tabebuia broadleef tree, Tabebuia avellanedae (also known as Tabebuia impetiginosa; see Brazilian medicobotanical encyclopedia (Yakuyou-Shokubutsu Jiten), p. 158 to 159, Aboc Co. Ltd., 1st issued on Apr. 27, 1996), is a mighty tree originally come from the South America's Amazon River basin. The plant is a traditional folk medicine known from the ancient Inca period, and a bark thereof has been used as a diuretic agent or an astringent. The plant has been focused as a medicinal resource with anticancer, antifungus, antibacterial, antiinflammatory activities and the like. Active ingredients containing the plant include NQ801 (chemical name: (S)-2-(1-hydroxyethyl)-5-hydroxynaphtho[2,3-b]furan-4,9-dione; NFD (registered mark); one of naphthoquinone natural colorants) of formula:

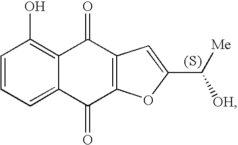

which has been known to have a significant anticancer activity as well as a potent cytotoxicity against various tumor cells (see Patent Documents 1, 2 and Nonpatent Document 1).

An extraction from barks of Tabebuia avellanedae has been generally known as a method for obtaining NQ801 (see Patent Document 1 and Nonpatent Document 1). However, since a cultivation of the plant has been difficult, and further, an extraction from barks thereof has given NQ801 in very low yields of 0.001 to around 0.05% by dry mass (see Nonpatent Document 1), it has been far from easy to mass-produce NQ801 in a conventional manner.

In contrast, a callus induction from plants has been known as one of tissue cultivation methods using a plant cell engineering technology (see Patent Documents 3 to 6). Callus is amorphous clumps of tissues obtained by incubating parts (explants or slices) of differentiated plant tissues in appropriate media, and may be differentiated into a complete plant.

Generally, a method for producing active ingredients of a plant by a callus culture has required the first step of inducing clumps of cells or callus from a plant to grow by subculturing plant cells in appropriate media, and the second step of producing active ingredients by transplanting the clumps of cells or callus grown in the first step to other fresh media to subculture (see Patent Documents 3 to 6 and Nonpatent Document 1). In other words, active ingredients have been produced by subculturing every 4 to 5 weeks through the generations in a cell-growth step, followed by replacing media for the cell-growth with other fresh media by conventional callus culture methods. Accordingly, such conventional callus culture methods have required enormous amounts of time and efforts to produce desired amounts of active ingredients contained in a plant.

### Patent Document 1: JP6-145162

### Patent Document 2: JP2006-290871

### Patent Document 3: JP5-186361

### Patent Document 4: JP7-184679

### Patent Document 5: JP10-42888

### Patent Document 6: JP10-52296

### Nonpatent Document 1: Shinichi UEDA et al., Phytochemistry, 1994, Vol. 36, No. 2, p. 323-325

### DISCLOSURE OF INVENTION

### Problems to be Resolved by the Invention

One of the problems to be resolved by the present invention is to efficiently produce an active ingredient, particularly an anticancer active ingredient, contained in Bignoniaceous plants of which a mass production is difficult in a conventional manner by using the modified cell-cultivation in the present invention.

Another problem to be resolved by the present invention is to reduce necessary steps for producing an active ingredient by controlling culture conditions and to stably and sustainably produce the active ingredient. The culture conditions particularly include media and various materials contained therein.

Further problem to be resolved by the present invention is to provide a food product (particularly, a health food or a functional food, etc.), a pharmaceutical or a pharmaceutical composition, comprising all or part of clumps of cells or callus produced by the modified cell cultivation in the present invention.

### Means of Solving the Problems

The present invention provides the following embodiments:

(1) A method for preparing an anticancer active ingredient, comprising an ingredient-production step (a) wherein an anticancer active ingredient is produced by suspension-culturing or solid-culturing clumps of cells of Bignoniaceous plant in one or more media;
(2) The method as described in (1), further comprising an ingredient-collection step (b) wherein one or more anticancer active ingredients are collected from the clumps of cells obtained in the step (a), the media used in the step (a) or both;
(3) The method as described in (1), wherein Bignoniaceous plant is Tabebuia avellanedae or Tabebuia impetiginosa;
(4) The method as described in (1), wherein the media includes a growth regulator and an organic complement;
(5) The method as described in (4), wherein the growth regulator is selected from the group consisting of auxin, cytokinin and a mixture thereof;
(6) The method as described in (5), wherein the auxin is selected from the group consisting of naphthaleneacetic acid (NAA), 2-naphthoxyacetic acid, indoleacetic acid (IAA), 4-chloroindoleacetic acid, indolebutyric acid (IBA), 2,4-dichlorophenoxyacetic acid (2,4-D) and a mixture thereof, and the cytokinin is selected from the group consisting of benzyladenine (BA), kinetin (K) and a mixture thereof;
(7) The method as described in (5), wherein the cytokinin is benzyladenine or kinetin;
(8) The method as described in (5), wherein both the auxin and the cytokinin are in a concentration of 0.1 to 2.5 ppm;
(9) The method as described in (4), wherein the organic complement is selected from the group consisting of saccharide, agar, gellan gum and a mixture thereof;
(10) The method as described in (1), wherein the anticancer active ingredient is 2-(1-hydroxyethyl)-5-hydroxynaphtho[2,3-b]furan-4,9-dione of formula:

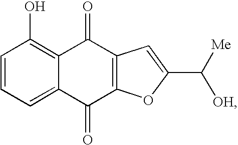

or a derivative thereof;
(11) The method as described in (1), wherein the anticancer active ingredient is NQ801 (chemical name: (S)-2-(1-hydroxyethyl)-5-hydroxynaphtho[2,3-b]furan-4,9-dione) of formula:

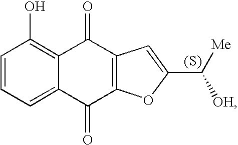

or a derivative thereof;
(12) The method as described in (1), wherein the anticancer active ingredient is produced at least 5% by mass of total mass of the clumps of cells obtained in the step (a), the media used in the step (a) or both;
(13) The method as described in (1), further comprising a cell-growth step wherein Bignoniaceous plant cells are grown by suspension-culture or solid-culture;
(14) The clumps of cells, obtained in the step (a) defined in (1); and
(15) A food product, comprising the clumps of cells obtained in the step (a) defined in (1), the media used in the step (a) or both.

The growth regulator in the ingredient-production step may affect an induction and a growth of the clumps of cells or callus of plant cells. Therefore, the induction and the growth of the clumps of cells or callus may depend on various factors such as types and concentrations of growth regulators.

### ADVANTAGEOUS EFFECTS OF THE INVENTION

The present invention shows nonconventional and advantageous effects where active ingredients can be efficiently produced only in an ingredient-production step under specific culture conditions. In other words, the present invention does not require a cell-growth step via subcultures through the generations, while conventional cell cultivation methods require said step. The present invention allows a material production in a shorter time (5 to 60 days) than that in a conventional manner. Certain selections of types or concentrations of growth regulators may increase the advantageous effect of the present invention.

The method of the present invention may further comprise a cell-growth step, and hence, the present invention also shows an additional advantageous effect where active ingredients can be produced many times by recycling (i.e. subculturing) the clumps of cells obtained in said step under specific culture conditions. This is because said step does not work against an active-ingredient production in the present invention since the clumps of cells grow simultaneously with the active-ingredient production by an optimization of culture conditions including growth regulators.

The present invention also shows an additional advantageous effect where a stable and sustained material production can be achieved in a shorter time than that of a conventional method wherein active ingredients are directly extracted from plant tissues of plants. This is because the present invention requires no time or very short time, even if needed, for an extra step necessary for callus induction and growth in a conventional manner.

Although Bignoniaceous plants contain various physiologically active ingredients, the present invention shows an unanticipated advantageous effect where a specific active ingredient, particularly NQ801, may be selectively produced as one of major ingredients.

An extract with an organic solvent from media used in the ingredient-production step of the present invention has a remarkable cytotoxic activity against tumor cells, which is caused by a secretion of active ingredients from the clumps of cells or callus which produce the active ingredients in said step.

### BRIEF DESCRIPTION OF DRAWINGS

FIG. 1 shows plant tissues in a sterilization study of Reference Example 2. In FIG. 1, α shows tissues sterilized by 70% ethanol only and β shows tissues sterilized by 70% ethanol and sodium hypochlorite.

FIG. 2 shows a medium one month after the incubation start on the medium containing naphthaleneacetic acid (NAA) and benzyladenine (BA) in a concentration of 1.0 ppm each in Example 2.

FIG. 3 shows suspension cultures (i.e. liquid cultures) in the ingredient-production step of Tabebuia avellanedae in Example 3.

FIG. 4 shows a medium in the quantitative analysis in Example 4.1.

FIG. 5 shows HPLC chart of an extract from the primary culture and a mixture of the extract and NQ801 in the ingredient-production system of Example 4.3.

FIG. 6 shows IT-TOF-MS spectrum of an extract from the primary culture in the ingredient-production system of Example 4.3. Each number over each peak means a molecular weight of a material of each peak, and underlined numbers mean peaks of NQ801 and an isomer thereof.

FIG. 7 shows HPLC chart of ingredients collected from the ingredient-production system wherein callus is cultured for 5 days in a medium containing 2,4-D and BA in a concentration of 1.0 ppm each. In FIG. 7, each numeric value over each peak means a retention time of each peak, an area ratio (%) of total ingredients contained in the medium, and an area value from beneath. The area ratio is calculated by UV rays (254 nm) in HPLC.

FIG. 8 shows HPLC chart of ingredients collected from the ingredient-production system wherein callus is cultured for 15 days in a medium containing 2,4-D and BA in a concentration of 1.0 ppm each. UV rays (254 nm) in HPLC indicate that NQ801 is contained in 15.8% of the total.

FIG. 9 shows HPLC chart of ingredients collected from the ingredient-production system wherein callus is cultured for 10 days in a medium containing 2,4-D and K in a concentration of 1.0 ppm each. A number over a peak shows a retention time.

FIG. 10 shows HPLC chart of ingredients collected from the ingredient-production system wherein callus is cultured for 20 days in a medium containing 2,4-D and K in a concentration of 1.0 ppm each.

FIG. 11 shows HPLC chart of ingredients collected from the ingredient-production system wherein callus is cultured for 30 days in a medium containing 2,4-D and K in a concentration of 1.0 ppm each. Each number over each peak shows a retention time.

FIG. 12 shows HPLC chart of extracts from the primary culture to the third generation subculture in the ingredient-production system of Example 4.3. It shows that relative area ratios of the produced NQ801 are almost constant even in subcultures.

FIG. 13 shows HPLC charts of ingredients collected from the ingredient-production systems wherein callus is suspension-cultured for 15, and 60 days in a medium containing NAA and BA in a concentration of 1.0 ppm each, and suspension-cultured for 30 days in a medium containing 2,4-D and BA in a concentration of 1.0 ppm each.

### BEST MODE FOR CARRYING OUT THE INVENTION

The method for preparation in the present invention comprises the ingredient-production step (a) and the ingredient-collection step (b).

In the ingredient-production step (a), slices of a plant, or callus induced therefrom by a callus culture is incubated in a medium to produce active ingredients.

The slices of a plant are collected from any sites of a plant. The plant includes Bignoniaceous plant, preferably Bignoniaceous Tabebuia plant, more preferably Tabebuia avellanedae or Tabebuia impetiginosa.

The sites of a plant from which slices of a plant are collected include tissues with growth points including leaves, stalks, roots, buds, petals, seeds, embryos, barks or a plant body, preferably leaves or stalks.

Callus culture means an incubation of parts (i.e. explants or slices) of differentiated plant tissues in appropriate media. Herein, amorphous clumps of tissues obtained by callus culture refer to “clumps of cells” or “callus”. The clumps of cells and callus include a mass of tissues which is formed in an initial stage of cell cultivation even if it is not clearly recognized to be formed in usual, and which makes media colored, preferably colored to yellow or purple-red. Preferably, the clumps of cells or callus are induced from plant tissues of Bignoniaceous plant, more preferably plant tissues of Bignoniaceous Tabebuia plant, particularly plant tissues of Tabebuia avellanedae or Tabebuia impetiginosa.

The media include one or more liquid or solid media wherein Murashige-Skoog (MS) medium or Linsmaier-Skoog (LS) medium, preferably Murashige-Skoog liquid medium, is contained as a basal medium. The media used herein comprises growth regulators and organic complements as well as a basal medium. The media may further comprise any other materials which do not inhibit a production of active ingredients. The materials include macro or micro salts or vitamin. A preferable pH of the media is 5 to 6, particularly 5.6. One or more growth regulators may be added to the media in the present invention.

The growth regulator means a generic name of agents which affect induction and growth of callus, and also refers to a phytohormone. A preferable growth regulator is selected from the group consisting of auxin, cytokinin and a mixture thereof. A concentration of the growth regulator is 0.1 to 2.5 ppm, preferably 0.5 to 2.0 ppm, more preferably 1.0 ppm.

The auxin is one of growth regulators, and means either naturally-occurring auxin or synthetic auxin. The naturally-occurring auxin includes an agent selected from the group consisting of indoleacetic acid (IAA), indolebutyric acid (IBA) and a mixture thereof. A preferable naturally-occurring auxin is indoleacetic acid, 4-chloroindoleacetic acid or indolebutyric acid. The synthetic auxin includes an agent selected from the group consisting of naphthaleneacetic acid (NAA), naphthoxyacetic acid, 2,4-dichlorophenoxyacetic acid (2,4-D) and a mixture thereof. A preferable synthetic auxin is naphthaleneacetic acid, 2-naphthoxyacetic acid or 2,4-dichlorophenoxyacetic acid, more preferably naphthaleneacetic acid or 2,4-dichlorophenoxyacetic acid.

The cytokinin is one of growth regulators. A preferable cytokinin is benzyladenine (BA) or kinetin (K).

The organic complement means an organic substance contained in a medium. A preferable organic complement is selected from the group consisting of saccharide, agar, gellan gum and a mixture thereof. A preferable organic complement in the suspension culture is saccharide, and that in the solid culture is a substance selected from the group consisting of agar, gellan gum and a mixture thereof. A preferable saccharide is disaccharide, particularly sucrose.

The macro or micro salt means an inorganic salt which may be contained in the media for plant cultivation. The inorganic salt includes a substance which is constituted by N, P, K, Ca, Mg, Mn, Zn, B, Co, Cu, Mo, Fe or I.

The vitamin means a conventional vitamin usually used in the art. A preferable vitamin is selected from the group consisting of vitamin B family: nicotinic acid, thiamine, pyridoxine, inositol and a water-soluble salt thereof.

The active ingredient means any physiologically active agents contained in a plant. A preferable active ingredient is one or more ingredients with cytotoxic, antitumor or anticancer activities. More preferable one is a naphthoquinone ingredient with a cytotoxic, antitumor or anticancer activity. Further preferable one is 2-(1-hydroxyethyl)-5-hydroxynaphtho[2,3-b]furan-4,9-dione of formula:

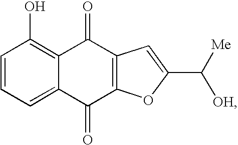

or a derivative thereof including a salt, hydrate or solvate thereof. Particularly preferable one is NQ801 (chemical name: (S)-2-(1-hydroxyethyl)-5-hydroxynaphtho[2,3-b]furan-4,9-dione; NFD (registered mark)) of formula:

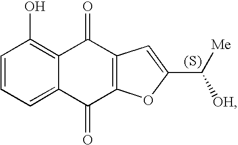

or a derivative thereof including a salt, hydrate or solvate thereof.

Both the ingredient-production and the material-production mean that active ingredients of a plant are produced by culturing callus which is induced from slices of a plant or plant tissues.

The culture period required for the ingredient-production step is 5 to 60 days, preferably 15 to 45 days. More preferable one is 30 to 45 days in case that benzyladenine is used as cytokinin, and 15 to 30 days in case that kinetin is used as cytokinin. The culture in the ingredient-production step may be either liquid culture (including suspension culture and static culture) or solid culture, preferably suspension culture or solid culture. The culture may be carried out in the primary culture, or the second or more generation subcultures.

In the ingredient-collection step (b), the active ingredient produced in the ingredient-production step is collected by an extraction from the ingredient-production system.

The active ingredient is usually released from the clumps of cells into media, and may be collected by an extraction from media. The active ingredient may be also collected by either a direct extraction from the clumps of cells or an extraction from a mixture of media and the clumps of cells.

The extraction is usually carried out with an organic solvent such as an alkyl halide solvent including chloroform after an optional addition of water to media or a mixture of media and the clumps of cells.

The ingredient-production system means a substance selected from the group consisting of the clumps of cells obtained in the ingredient-production step, preferably those which produce active ingredients therein, the media used therein and a mixture thereof.

The method for preparation in the present invention may further comprise the aforesaid callus culture stage as the cell-growth step. In the cell-growth step, callus may be induced from a plant to grow, followed by cultured in the ingredient-production step.

The conditions including media and culturing methods used in the cell-growth step may be the same as those in the ingredient-production step or different therefrom.

The culture period required for the cell-growth step is 0 to 60 days, preferably 30 to 45 days.

In the conventional technology, separate media are used for the cell-growth and the ingredient-production. Specifically, callus does not produce desired ingredients in a conventional manner without replacing a medium for the cell-growth with another medium for the ingredient-production after subcultures for several generations every 4 to 5 weeks. In other words, enormous amounts of time and efforts are required for eventually obtaining desired ingredients in a conventional manner.

In contrast, the same medium may be used in the present invention through callus formation to the material-production by using a medium comprising a combination of 2,4-D and BA, NAA and BA, or 2,4-D and K (see Tables 2 and 3). In other words, since a conventionally used subculture of callus may not be essential in the cell-growth step in the present invention, the present invention shows an unanticipated effect that callus formation and the material-production may be performed in the ingredient-production step even without the cell-growth step.

The present invention also shows a remarkable advantageous effect that active ingredients may be produced in incredibly earlier stage than that in a conventional callus formation or an ingredient-production after a conventional subculture. In other words, the present invention shows an advantageous effect that desired ingredients may be obtained with saving the effort to grow callus by subculture and subsequently to transfer the grown callus to a medium for the ingredient-production.

The present invention is also characterized by callus which is allowed to be subcultured as well as being cultured from the callus formation to the material production in a single medium containing NAA and BA (see Tables 2 and 3). In this respect, since active ingredients may be extracted from a medium obtained after callus subculture and further extracted by transferring the subcultured callus into another fresh medium in the present invention, the present invention may achieve a stable and continuous preparation of active ingredients.

The plant used in the ingredient-production step and the cell-growth step may be obtained by cultivating from seeds thereof.

The cultivation of a plant may be preferably performed in a greenhouse at 25 to 35° C. under conditions including light period, specifically dark period 8 hours and light period 16 hours. A grown plant may be used after sterilization in the ingredient-production step or the cell-growth step.

The plant may be sterilized by alcohol containing a strong alkaline salt, preferably by a mixture of 60 to 80% ethanol and 1% sodium hypochlorite for 5 to 10 minutes. More preferably, the plant may be sterilized by a mixture of 70% ethanol and 1% sodium hypochlorite for 10 minutes.

The ingredient-production system herein may be directly (for example, without processing) or indirectly (for example, in the form of a powder or liquid) contained in a food product, pharmaceutical or pharmaceutical composition as the active ingredient or another component.

### EXAMPLES

### Reference Example 1

### Cultivation of Plant

Tabebuia avellanedae (Tabebuia avellanedae seeds are available from Taheebo Japan Co., Ltd.; Tabebuia avellanedae seed (Lot: 000717)) was cultivated in order to be used in the present invention. An 1% of a nutrient-free agar medium was used as a medium for seeding. The plant was germinated in about one week, and then naturalized in horticultural soil (red soil 7:vermiculite 3) to be grown at 25° C. for 16 hours in light period and 8 hours in dark period in a climate chamber. Tabebuia avellanedae, which was originally come from the South America's Amazon River basin, was grown like bine at the growth points as the temperature was increased to 28° C. and the humidity was increased by a spray bottle. After growing to some extent, the plant was grown in a simple greenhouse in an incubator. As the temperature was increased to the ground temperature (30° C.) and the humidity was increased in the simple greenhouse, the plant was grown and leaves and stalks thereof were developed.

### Reference Example 2

### Sterilization Study

A sterilization of the plant grown in Reference Example 1 was studied. A solvent for sterilization was optimized among two different kinds of solvents such as 70% ethanol (available from Wako Pure Chemical Industries, Ltd.) only, or a combination of 70% ethanol and 1% sodium hypochlorite solution (available from Wako Pure Chemical Industries, Ltd.).

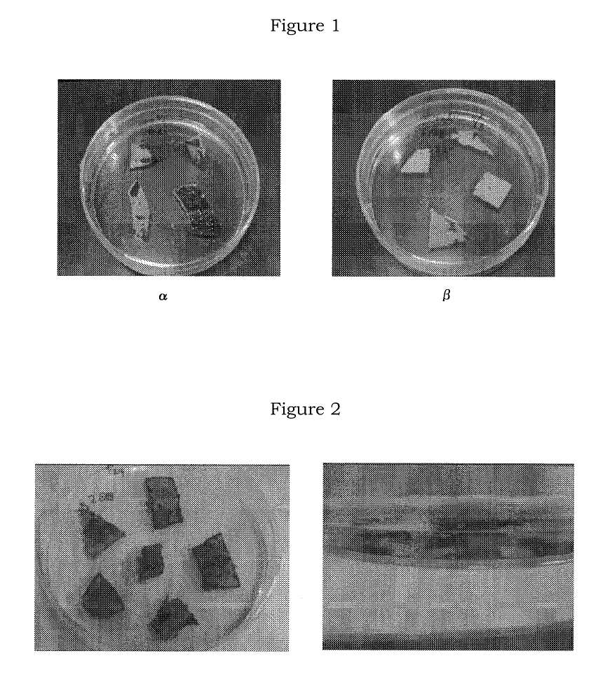

Specifically, the sterilization was performed in a clean bench by (α) 70% ethanol only for 2 minutes or by (β) a combination of 70% ethanol for 1 minute and sodium hypochlorite for 10 minutes (see FIG. 1). The plants sterilized by 70% ethanol only were killed 2 days after the sterilization, while no plant death was observed in those sterilized by a combination of 70% ethanol and sodium hypochlorite. Since the result showed that the sterilization β was desirable, the plants were sterilized in the manner of β in the following Examples.

The plants sterilized in the above manner were washed with a sterilized water several times, and then leaf discs thereof (leaves: about 1 cm², stalks: 1-cm sections) were prepared from the plants to be used in the subsequent Examples.

### Example 1

### Medium Screening in the Cell-Growth Step

Murashige-Skoog (MS) medium in basal compositions shown in Table 1 was used as a medium, and the culture conditions were at 30° C. and 24 hours in dark period.

TABLE 1

| MS Medium | Basal Compositions |
|---|---|
| Constituents | mg/l |
| Sucrose | 10000 |
| Inorganic Salts | 2150 |
| Vitamin | 1 |
| pH | 5.6 |

In order to study callus formation levels and growth and development conditions in terms of callus formation and growth, types and concentrations of growth regulators were studied.

The growth regulators added to a medium in the Example were selected from naphthaleneacetic acid (hereinafter referred to as NAA), benzyladenine (hereinafter referred to as BA), kinetin (hereinafter referred to as K) or indoleacetic acid (hereinafter referred to as IAA) which generally accelerated callus formation of slices of Tabebuia avellanedae, or 2,4-dichlorophenoxyacetic acid (hereinafter referred to as 2,4-D) which accelerated an extraction of a naphthoquinone ingredient, and a combination of auxin and cytokinin was used in the screening assay. Each concentration of each growth regulator was 0.5, 1.0 or 2.0 ppm.

TABLE 2
Concentration Study of Growth Regulator
in the Cell-Growth Step (30 Day-Culture)

| | NAA + BA | IAA + K | 2,4-D + BA | 2,4-D + K |
|---|---|---|---|---|
| 0.5 ppm | Δ | ◯ | X | Δ |
| 1.0 ppm | Δ | ◯ | X | X |
| 2.0 ppm | Δ | ◯ | X | X |

◯: Callus was formed, and callus clusters (clumps of cells) continued to grow by the day.
Δ: Callus was formed, and callus clusters (clumps of cells) grew slowly.
X: Callus was formed, and callus clusters (clumps of cells) turned black without growing.

In the cell-growth step, neither callus was formed nor active ingredients were produced in a medium without any growth regulators.

As shown in Table 2, callus clusters grew slowly in a medium containing a growth regulator consisting of NAA and BA, while callus clusters grew faster in a medium containing a growth regulator consisting of IAA and K than other media.

On the other hand, callus was formed but callus clusters turned black in a medium containing a growth regulator consisting of 2,4-D and BA.

In a medium containing a growth regulator consisting of 2,4-D and K, callus was formed faster and callus clusters turned black earlier than other media.

### Example 2

### Medium Screening in Ingredient-Production Step

### Example 2.1

### Growth Regulator Concentration Study

Once a naphthoquinone ingredient produced from callus started to mix in a medium, the medium turned yellow. Naphthoquinone ingredient-containing concentrations in the medium could be visually observed for results. In terms of material production, types and concentrations of growth regulators, culture periods and combinations of growth regulators from callus formation to naphthoquinone ingredient-extraction were studied.

TABLE 3
Concentration Study of Growth Regulator in
Ingredient-Production Step (30 Day-Culture)

| | NAA + BA | IAA + K | 2,4-D + BA | 2,4-D + K |
|---|---|---|---|---|
| 0.5 ppm | B | C | A | B |
| 1.0 ppm | A | C | A | A |
| 2.0 ppm | B | C | A | A |

A: A medium before extraction colored saffron yellow.
B: Yellow color of a medium before extraction could be observed on white paper.
C: Clear and colorless (organic solvent layer after extraction did not color yellow).

In the ingredient-production step, no productions of naphthoquinone ingredients could be observed in a medium without any growth regulators.

Tables 2 and 3 show that although callus formed turned black, NQ801 or a derivative thereof was efficiently produced in the ingredient-production step in a medium containing a combination of 2,4-D and BA, or 2,4-D and K as a medium for both cell-growth and ingredient-production compared to other media;

Further, callus formed faster but no NQ801 was produced in a medium containing IAA and K (see Tables 2 and 3).

### Example 2.2

### Culture Period Study

TABLE 4
Culture Period Study in Ingredient-Production Step

| | NAA + BA | IAA + K | 2,4-D + BA | 2,4-D + K |
|---|---|---|---|---|
| 15/day | B | C | A | A |
| 30/day | A | C | A | A |
| 45/day | A | C | A | — |
| 60/day | B | C | A | — |

A: A medium before extraction colored saffron yellow.
B: Yellow color of a medium before extraction could be observed on white
paper.
C: Clear and colorless (organic solvent layer after extraction did not color
yellow).
—: Uninvestigated.

As shown in Table 4, a production of naphthoquinone ingredients was changed depending on culture periods only in a medium containing a growth regulator consisting of NAA and BA.

Saffron yellow ingredients were extracted from a medium containing a growth regulator consisting of 2,4-D and BA, and 2,4-D and K. Naphthoquinone ingredients started to be produced in these media 5 to 10 days after incubation.

Saffron yellow ingredients, i.e. naphthoquinones, were produced in a medium containing a growth regulator consisting of 2,4-D and K even 20 days after incubation.

### Example 2.3

### Subculture Study

A plant was cultured in a medium containing NAA and BA in a concentration of 1.0 ppm, respectively. One month after starting culture, a cultured cell was subcultured in fresh medium consisting of the same compositions as that in the primary culture (see FIG. 2). Once callus formed, a medium turned yellow, which it was considered that naphthoquinone ingredients were produced. It shows that callus can be subcultured in the above medium in addition to the fact that the same medium can be used through callus formation to material production (see Tables 2 and 3).

### Example 3

### Suspension Culture in Ingredient-Production Step

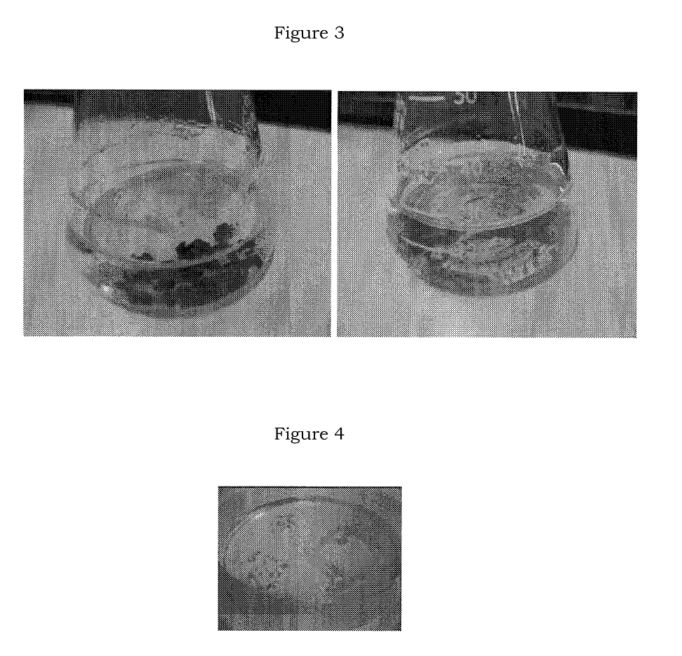

Callus derived from Tabebuia avellanedae, by which yellow ingredients were produced in the cell-growth step, was cultured in MS liquid medium. An extraction of naphthoquinone ingredients from callus body by suspension culture of callus was studied. A combination of NAA and BA in a concentration of 1.0 ppm each was used as the growth regulator. A medium turned yellow one week after starting culture (see FIG. 3). The medium was extracted with chloroform, concentrated to give naphthoquinone ingredients (20 mg) in liquid medium (150 ml). Even in a repetitive manner, the medium did not color after one-month culture.

### Example 4

### Extraction (Ingredient-Collection Step) and Analysis of Naphthoquinone Ingredients from Ingredient-Production System

### Example 4.1

### Quantitative Analysis

Once a material which was considered to be one of naphthoquinone ingredients produced in the ingredient-production system was incorporated in a medium, the medium turned yellow (see FIG. 4). The material was identified by thin layer chromatography (TLC) analysis.

First, to a medium (200 ml) in vessel was added chloroform (300 ml), and then the desired ingredient was extracted from the medium with chloroform. The extract was aspirated to filter and concentrated by evaporator. Then, a mass of the obtained naphthoquinone ingredient is 14 mg.

### Example 4.2

### TLC Analysis

Next, two spots of NQ801 and the extract from the ingredient-production system were put on TLC plate (Merck silica gel 60F 254 plate) and separated, by using normal-hexane:ethyl acetate (7:3) as a developing solvent.

A mixture of ingredients produced in the ingredient-production system was concentrated, and then spotted on TLC plate several times. The separated spot was detected by UV (UV IRRADIATER(SOGO LABORATORY GLASS WORKS CO., LTD); UV rays 254 nm), and both an ingredient which was in the same position as the pure NQ801 and several kinds of other ingredients around the spot could be observed in the ingredients produced in the ingredient-production system.

### Example 4.3

### High Performance Liquid Chromatography (HPLC) Analysis

Concentrated fractions of the primary culture ingredients in the ingredient-production system were analyzed by HPLC, and HPLC patterns thereof were compared with those of NQ801.

The analysis was performed by LC-10A HPLC system manufactured by SHIMADZU CORPORATION.

Concentrated extracts from the primary culture ingredients (NAA+BA 1.0 ppm, one month-culture period) and a mixture of the extracts and NQ801 were analyzed by HPLC.

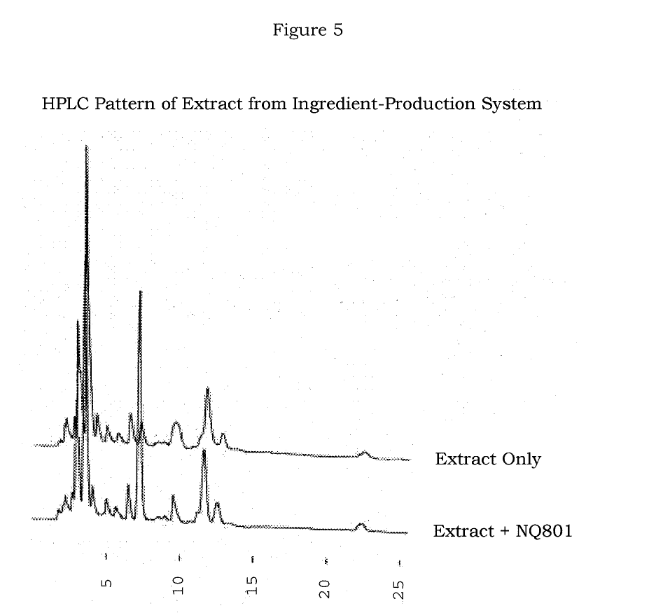

As shown in FIG. 5, NQ801 was found in the ingredients produced in the ingredient-production system.

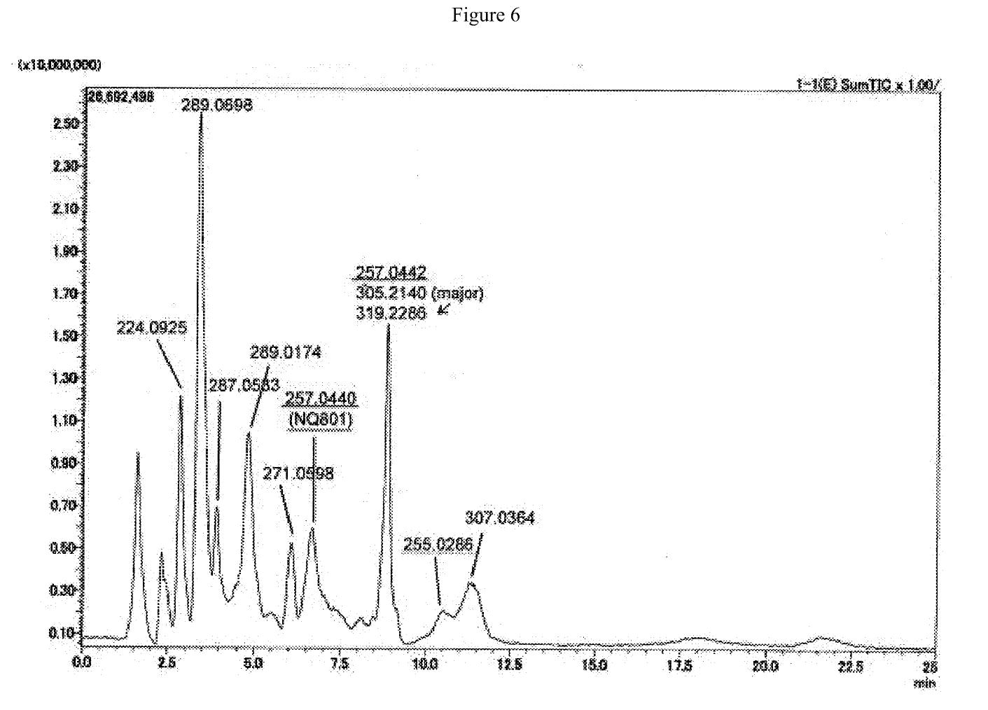

FIG. 6 shows an analytical result of concentrated extracts from the primary culture ingredients by IT-TOF-MS (LCMS-IT-TOF manufactured by SHIMADZU CORPORATION). Though there were several other peaks which showed a similar molecular weight to NQ801 other than NQ801 itself, NQ801 was, surprisingly, selectively obtained as one of the main products according to the preparation of the present invention.

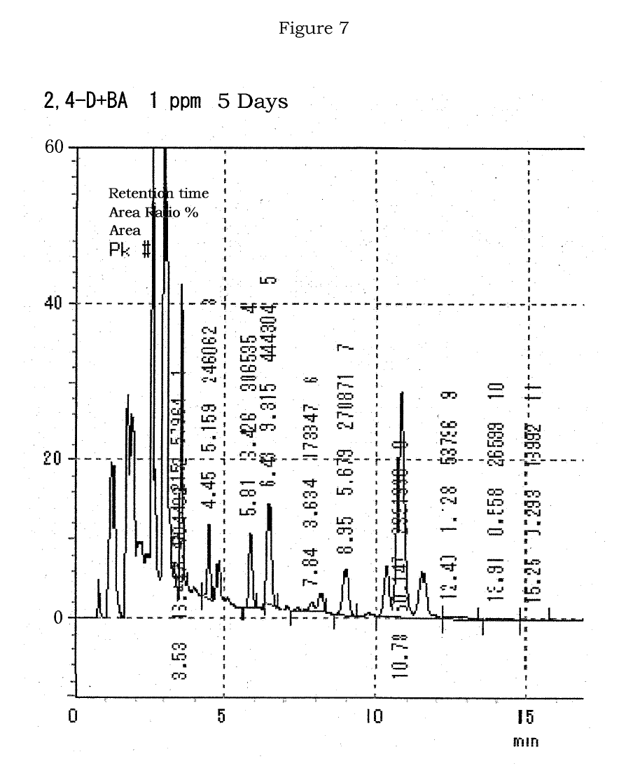

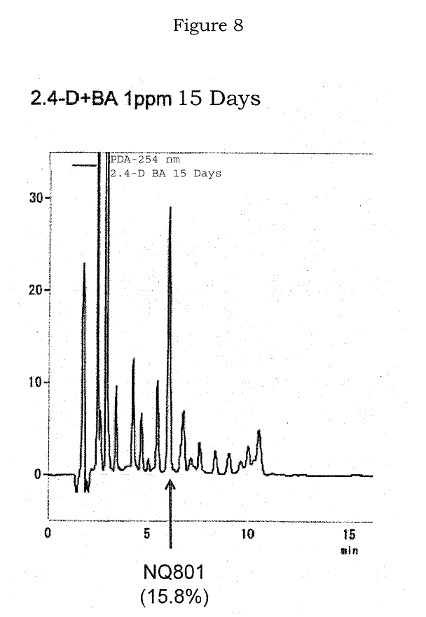

Calculations from area ratios of peaks obtained by UV rays (254 nm) in HPLC show that clumps of cells or callus obtained in the ingredient-production system contain over 5 to 15% by mass of NQ801 of total mass of the system (see FIGS. 7 and 8).

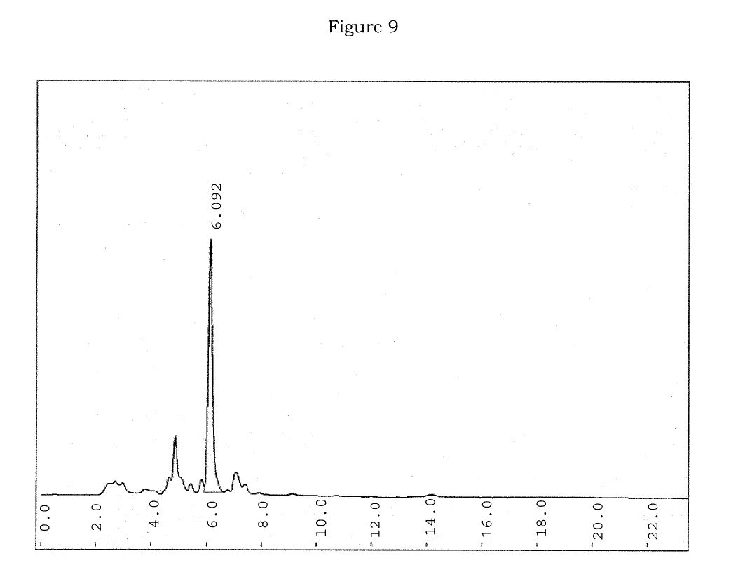

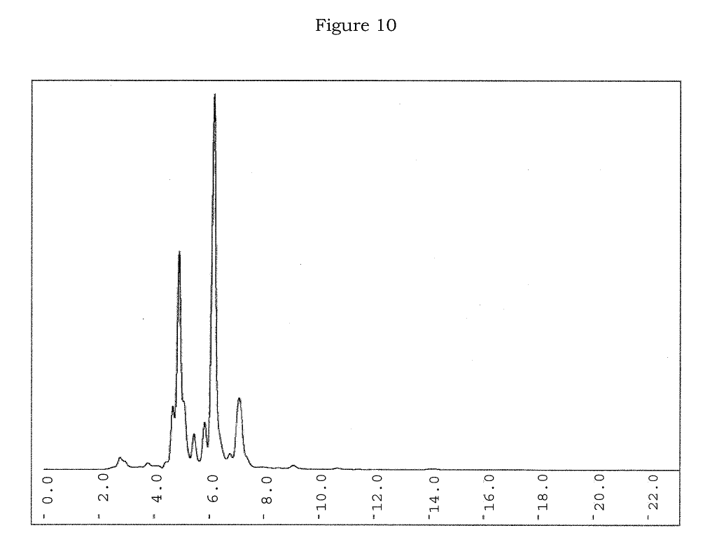

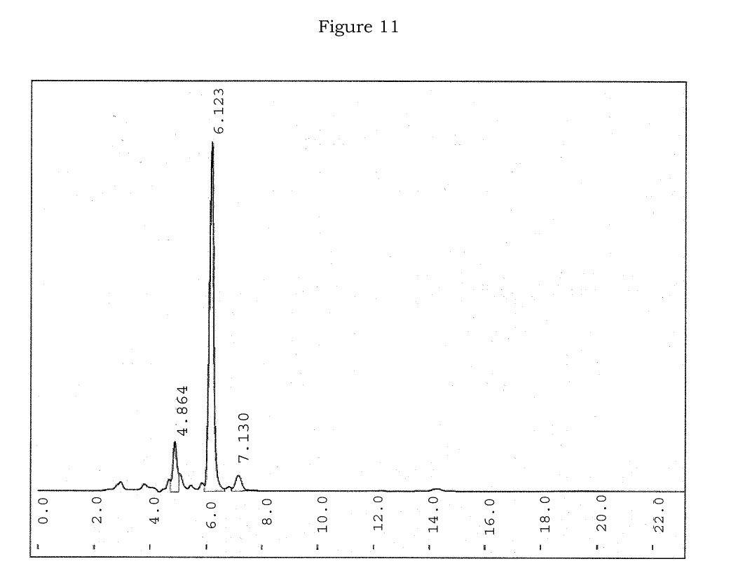

Particularly, a medium containing 2,4-D and BA turned yellow a few days after incubation of parts (plant slices) of plant tissues. As shown in FIG. 7, NQ801 was confirmed to be produced in about 9.3% as area ratios to total ingredients 5 days after incubation. A 15 day culture in a medium containing 2,4-D and BA in a concentration of 1.0 ppm gave 15.8% of NQ801 to area ratios of total ingredients (see FIG. 8). After 10 day- and 30 day-incubation, NQ801 was confirmed to be produced in a medium containing 2,4-D and K in 50% and 80% as area ratios to total ingredients, respectively (see FIGS. 9 and 11).

The results show that NQ801 or derivatives thereof are considered to be produced in these media without any clear callus formation. NQ801 is extracted below 0.001% by mass from Tabebuia avellanedae barks in a conventional manner, while NQ801 is found to be produced in an unanticipated and significant amount in the preparation of the present invention.

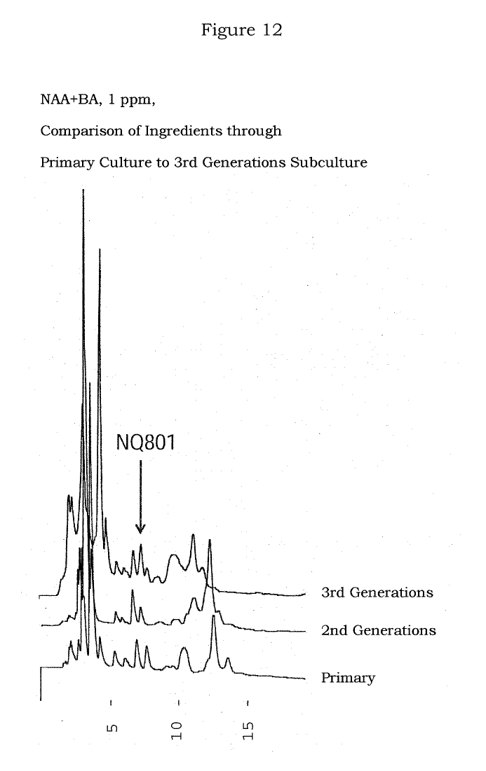

Next, HPLC patterns of concentrated extracts were compared from the primary culture to the third generation subculture. The result is shown in FIG. 12.

The primary culture was carried out for one-month culture period, and the second and the third generations were cultured for the additional 5 day culture, respectively. Mass of naphthoquinone ingredients after concentrations of the primary culture, the second generation subculture and the third generation subculture was 14, 15 and 15 mg, respectively.

Peak area ratios of naphthoquinone ingredients to total ingredients in the ingredient-production system were almost constant through the primary culture to the third generation subculture. Accordingly, the Example shows that callus subculture is unnecessary in the ingredient-production system in the present invention.

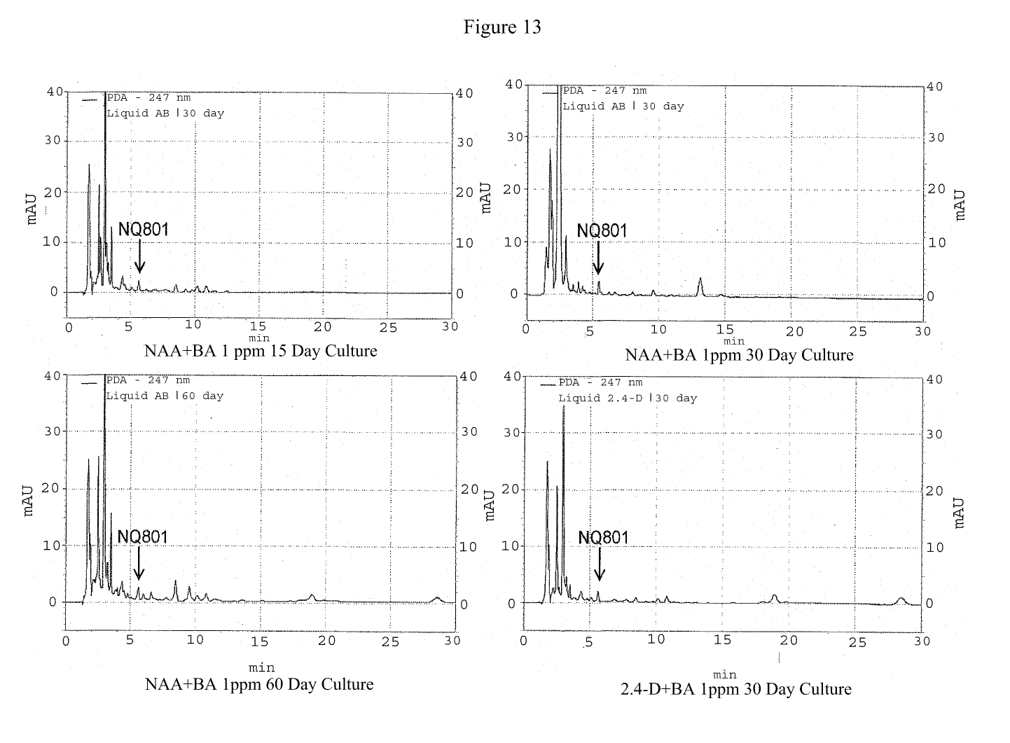

For a suspension culture in the ingredient-production step, ingredients in the ingredient-production system were determined by HPLC in a medium containing NAA and BA in a concentration of 1.0 ppm and a medium containing 2,4-D and BA in a concentration of 1.0 ppm. FIG. 13 shows HPLC chart. As shown in FIG. 13, NQ801 productions were observed in all culture conditions.

### Cytotoxicity Experiment

An extract from a medium in the ingredient-production step in the present invention was studied in a cytotoxicity experiment. Specifically, Experiments 1 and 2 were carried out as follows.

Experiment 1 Anticancer Activity against A-549 Cells (Lung Cancer Cells)

A-549 cells (lung cancer cells; Strain No. ECACC86012804; available from Laboratory Products Dept. of Dainippon Pharmaceuticals Inc. (now Dainippon Sumitomo Pharma Co., Ltd.)) were preliminarily seeded in 35 mm petri dishes containing Dulbecco's modified Eagle medium (DMEM) containing 20% of fetal bovine serum (FBS) in a concentration of 1×10⁵/ml. The seeded cells were incubated at 36° C. for 1 day under 5% CO₂ and were confirmed to be stuck at the bottom of the petri dishes. The resulting A-549 cells were divided into three groups, to the first group of which were added extracts from a medium used in the ingredient-production system in concentrations of 1, 10, 100 and 1000 μg/ml, to the second group of which was added NQ801 which was known to have an anticancer activity (see Patent Document 2) in said concentrations, and to the third group of which were added ether fractions extracted from Tabebuia avellanedae barks in a conventional manner in said concentrations. These groups were incubated at 36° C. for 3 days, and viable cells were counted and viabilities were calculated. The result is shown in Table 5.

Experiment 2 Anticancer Activity against MCF-7 Cells (Breast Cancer Cells)

Cytotoxicity experiments for MCF-7 cells (breast cancer cells; Strain No. ECACC86012803; available from Laboratory Products Dept. of Dainippon Pharmaceuticals Inc. (now Dainippon Sumitomo Pharma Co., Ltd.)) were carried out in the similar manner to the Experiment 1. The result is shown in Table 5.

TABLE 5
Cytotoxicity Experiments

| | | | |
|---|---|---|---|
| 1000 μg/ml | 100 μg/ml | 10 μg/ml | 1 μg/ml |

Cell Viabilities (%) of A-549 (Lung Cancer Cells)

| | | | | |
|---|---|---|---|---|
| Extracts from | 0 | 40 | 80 | 100 |
| Medium | | | | |
| NQ801 | 0 | 30 | 60 | 90 |
| Ether Fractions | 70 | 100 | 100 | — |

Cell Viabilities (%) of MCF-7 (Breast Cancer Cells)

| | | | | |
|---|---|---|---|---|
| Extracts from | 0 | 20 | 70 | 100 |
| Medium | | | | |
| NQ801 | 0 | 20 | 60 | 90 |
| Ether Fractions | 60 | 80 | 100 | — |

Table 5 shows that extracts from a medium used in the ingredient-production system have comparable anticancer activities against A-549 cells to NQ801 in concentrations of 1, 10 and 100 μg/ml.

Table 5 also shows that extracts from a medium used in the ingredient-production system have comparable anticancer activities against MCF-7 cells to NQ801 in concentrations of 1 and 10 μg/ml and consistent anticancer activity to NQ801 in a concentration of 100 μg/ml.

Experiments 1 and 2 conclude that even though NQ801 is not isolated or purified from extracts from a medium used in the ingredient-production system, the extracts themselves have comparable anticancer activities to NQ801.

### INDUSTRIAL APPLICABILITY

Since extracts from a medium used in the ingredient-production system in the present invention have cytotoxic, antitumor and anticancer activities, they themselves may be utilized in medical, pharmaceutical, food and biochemical fields, etc. The ingredient-production system in the present invention is considered to contain multiple ingredients taking HPLC patterns thereof into consideration, and may be comprised as the active ingredient or another ingredient in a composition or food product without processing or in the form of a powder or liquid.

What is claimed is:

1. A method for preparing an anticancer active ingredient, comprising an ingredient-production step (a) wherein an anticancer active ingredient is produced by suspension-culturing or solid-culturing clumps of cells of Bignoniaceous plant in one or more media.

2. The method as claimed in claim 1, further comprising an ingredient-collection step (b) wherein one or more anticancer active ingredients are collected from the clumps of cells obtained in the step (a), the media used in the step (a) or both.

3. The method as claimed in claim 1, wherein Bignoniaceous plant is Tabebuia avellanedae or Tabebuia impetiginosa.

4. The method as claimed in claim 1, wherein the media includes a growth regulator and an organic complement.

5. The method as claimed in claim 4, wherein the growth regulator is selected from the group consisting of auxin, cytokinin and a mixture thereof.

6. The method as claimed in claim 5, wherein the auxin is selected from the group consisting of naphthaleneacetic acid (NAA), 2-naphthoxyacetic acid, indoleacetic acid (IAA), 4-chloroindoleacetic acid, indolebutyric acid (IBA), 2,4-dichlorophenoxyacetic acid (2,4-D) and a mixture thereof, and the cytokinin is selected from the group consisting of benzyladenine (BA), kinetin (K) and a mixture thereof.

7. The method as claimed in claim 5, wherein both the auxin and the cytokinin are in a concentration of 0.1 to 2.5 ppm.

8. The method as claimed in claim 4, wherein the organic complement is selected from the group consisting of saccharide, agar, gellan gum and a mixture thereof.

9. The method as claimed in claim 1, wherein the anticancer active ingredient is 2-(1-hydroxyethyl)-5-hydroxynaphthol-2,3-bifuran-4,9-dione of formula: 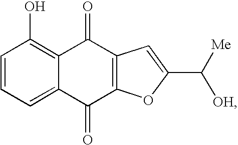
or a derivative thereof.

10. The method as claimed in claim 1, wherein the anticancer active ingredient is NQ801 (chemical name: (S)-2-(1-hydroxyethyl)-5-hydroxynaphtho[2,3-b]furan-4,9-dione) of formula: 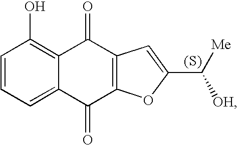
or a derivative thereof.

11. The method as claimed in claim 1, wherein the anticancer active ingredient is produced at least 5% by mass of total mass of the clumps of cells obtained in the step (a), the media used in the step (a) or both.

12. The method as claimed in claim 1, further comprising a cell-growth step wherein Bignoniaceous plant cells are grown by suspension-culture or solid-culture.

13. The clumps of cells, obtained in the step (a) defined in claim 1.

14. A food product, comprising the clumps of cells obtained in the step (a) defined in claim 1, the media used in the step (a) or both.
